(19) United States
(12) Patent Application Publication
(10) Pub. No.: US 2021/0370071 A1
(43) Pub. Date: Dec. 2, 2021

(54) SAFE DIRECT CURRENT STIMULATOR DESIGN FOR REDUCED POWER AND INCREASED RELIABILITY

(71) Applicant: THE JOHNS HOPKINS UNIVERSITY, Baltimore, MD (US)

(72) Inventor: Gene Yevgeny Fridman, Baltimore, MD (US)

(21) Appl. No.: 16/629,168

(22) PCT Filed: Jul. 9, 2018
(86) PCT No.: PCT/US2018/041246

§ 371 Date: Jan. 7, 2020

Related U.S. Application Data
(60) Provisional application No. 62/529,611, filed on Jul. 7, 2017.

(51) Int. Cl. A61N 1/36; A61N 1/20; A61N 1/378

(52) U.S. Cl. CPC A61N 1/36157; A61N 1/378; A61N 1/20

(57) ABSTRACT

Current state of the art neural prosthetics, such as cochlear implants, spinal cord stimulators, and deep brain stimulators use implantable pulse generators (IPGs) to excite neural activity. Inhibition of neural firing is typically indirect and requires excitation of neurons that then have inhibitory projections downstream. The present invention is directed to a safe direct current stimulator (SDCS) technology that is designed to convert electronic pulses delivered to electrodes embedded within an implantable device to ionic direct current (iDC) at the output of the device. iDC front the device can then control neural extracellular potential with the intent of being able to not only excite, but also inhibit and sensitize neurons, thereby greatly expanding the possible applications of neuromodulation therapies and neural interface mechanisms. The device of the present invention is designed to reduce power consumption by a factor of 12 and to improve its reliability by a factor of 8.

### CROSS REFERENCE TO RELATED APPLICATIONS

This application claims the benefit of U.S. Provisional Patent Application No. 62/529,611 filed Jul. 7, 2017, which is incorporated by reference herein, in its entirety.

### GOVERNMENT RIGHTS

This invention was made with government support under R01NS092726 awarded by the National Institutes of Health. The government has certain rights in the invention.

### FIELD OF THE INVENTION

The present invention relates generally to medical devices. More particularly, the present invention relates to a safe direct current stimulator design for reduced power and increased reliability.

### BACKGROUND OF THE INVENTION

Safe Direct Current Stimulator (SDCS) technology is being designed to create a new class of bioelectronic prostheses that excite, inhibit, and modulate the sensitivity of neurons. The term “safe” in the name implies only the intended safety within the device itself in avoiding electrochemical reactions to achieve ionic direct current (iDC) output. Vestibular implants, cochlear implants, and essentially all other chronically implantable neuroelectronic prostheses rely on charge-balanced, biphasic pulses or other forms of alternating current (AC) to excite neural or muscular activity without driving electrochemical reactions that would otherwise liberate toxic substances at the electrode-saline interface. Inhibition is difficult to achieve with these devices, because the need to avoid a net charge flow above a small threshold (e.g., ˜100 μC/cm2 electrode area for platinum electrodes) mandates the use of brief, charge-balanced pulses for which the cathodic, excitatory phase dominates the neural response. High frequency stimulation (2-20 kHz) has shown promise in being able to block neural activity, but has had challenges associated with large onset excitation and high power consumption.

In contrast to the anodic phase of a brief biphasic stimulus pulse, continuous low amplitude anodic iDC delivered by an extracellular electrode is effective at inhibiting neural activity. Continuous low amplitude cathodic iDC can excite neural activity in a graded, stochastic fashion unlike the phase-locked, more artificial behavior elicited by pulsatile stimuli. At reduced amplitudes, iDC stimulation can increase or decrease neural sensitivity to synaptic transmission. At higher amplitudes, iDC can be used to achieve complete nerve block. Given these advantages, DC has long been a mainstay of laboratory experiments, in which the charge-balance constraints imposed on medical devices can be ignored or overcome through the use of electrodes that are incompatible with chronic implantation. Chronically delivering DC stimulation via metal electrodes in the body is toxic because of gas generation by electrolysis, Faradaic charge transfer and corrosion. To avoid these reactions, the SDCS uses a rectification system within the device to convert alternating charge balanced pulses delivered to metal electrodes within the device to ionic direct current at the output of the device. However, the SDCS can be plagued by high power consumption and failure of one of the eight mechanical valves.

Accordingly, there is a need in the art for a safe, direct-current stimulator design for reduced power and increased reliability.

### SUMMARY OF THE INVENTION

The foregoing needs are met, to a great extent, by the present invention which provides a device for safe direct current stimulation of tissue including an actuator and a pair of current sources (I1, I2) engaged to apply current by the actuator. The device includes a pair of valves (V1, V2) operated in tandem by the actuator. The device also includes a first pair of electrodes (E1 and E2) and a second pair of electrodes (E1′ and E2′). The device is configured to operate in three stages (S1, S2, and S3). During S1, I1 drives current through the tissue via E1 and E1′ and I2 discharges E2 and E2′. During S2, the valves V1 and V2 change states, such that both valves are first opened and then one of V1 and V2 is closed in sequence. During S3, I2 drives current through the tissue and I1 discharges electrodes E1 and E1′.

In accordance with an aspect of the present invention, the device includes a first microcatheter tube filled with an electrolyte gel disposed on one side of the tissue and a second microcatheter tube filled with an electrolyte gel disposed on a second side of the tissue. The actuator can be formed from one NiTiNol wire. The NiTiNol wire is energized for half of a cycle to drive the pair of valves. The device includes microfluidic channels filled with an electrolyte. The microfluidic channels connect the first pair of electrodes with one of the pair of current sources and wherein the microfluidic channels connect the second pair of electrodes with the other one of the pair of current sources. The contacts of electrodes of the first and second pairs of electrodes take the form of a capacitor/resistor parallel pair with a series resistor. The contacts for the electrodes are 10 μF in parallel with a 2 MΩ resistor that are then in series with a 100Ω resistor. Opening and closing the pair of valves in tandem in state S2 requires 600 ms. The pair of current sources are configured to drive current while the pair of valves change state during S2 such that current is maintained through the tissue during the change of state. Each one of the pair of current sources is configured to drive 1 mA in a positive direction for four seconds and discharge at 4 mA over one second, such that a charge balance is maintained. Ionic current is directed to the tissue while a charge balance is maintained at the first pair of electrodes and the second pair of electrodes. The pair of current sources alternately deliver current pulses to the first and second pair of electrodes, such that alternating current pulses are converted to ionic direct current.

In accordance with another aspect of the present invention, a system for safe direct current stimulation of tissue includes an actuator. The system includes a pair of current sources (I1, I2) engaged to apply current by the actuator. The system also include a pair of valves (V1, V2) operated in tandem by the actuator. Additionally, the system includes, a first pair of electrodes (E1 and E2) and a second pair of electrodes (E1′ and E2′). The device is configured to operate in three stages (S1, S2, and S3). During S1, I1 drives current through the tissue via E1 and E1′ and I2 discharges E2 and E2′. During S2, the valves V1 and V2 change states, such that both valves are first opened and then one of V1 and V2 is closed in sequence. During S3, I2 drives current through the tissue and I1 discharges electrodes E1 and E1′. The system also includes a control system configured to monitor system output and direct the pair of current sources to compensate for irregularities in output.

In accordance with still another aspect of the present invention, the control system is configured to adjust the pair of current sources during a current driving phase. The control system is configured to integrate output of the pair of current sources to determine an exact charge delivered to the first and second pairs of electrodes. The control system is configured to discharge amplitude of a charge to account for a total accumulated charge. A first microcatheter tube filled with an electrolyte gel is disposed on one side of the tissue, and a second microcatheter tube filled with an electrolyte gel is disposed on a second side of the tissue. The actuator comprises one NiTiNol wire. The system includes microfluidic channels filled with an electrolyte, wherein the microfluidic channels connect the first pair of electrodes with one of the pair of current sources and wherein the microfluidic channels connect the second pair of electrodes with the other one of the pair of current sources. Contacts of electrodes of the first and second pairs of electrodes take the form of a capacitor/resistor parallel pair with a series resistor.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIGS. 1A and 1B illustrate a schematic diagram of a conceptual SDCS design with two states of the same device. FIG. 1C illustrates a schematic diagram that shows interruptions to output current flow during valve operation.

FIG. 2 illustrates a schematic diagram of two system design that uses one system (driven by I1 shown) to drive the current through the tissue, while the second system (driven by I2) to switch the valve states without causing output current interruption.

FIG. 3 illustrates a schematic diagram of three states of the present invention.

FIG. 4 illustrates a schematic diagram of an electrical equivalent component model of the present invention.

FIGS. 5A and 5B illustrate graphical views of modeling of the present invention. FIG. 5A shows 18 seconds of simulation. FIG. 5B expands the time between 3 and 7 seconds to illustrate the details of the switching sequence time course.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

Current state of the art neural prosthetics, such as cochlear implants, spinal cord stimulators, and deep brain stimulators use implantable pulse generators (IPGs) to excite neural activity. Inhibition of neural firing is typically indirect and requires excitation of neurons that then have inhibitory projections downstream. The present invention is directed to a safe direct current stimulator (SDCS) technology that is designed to convert electronic pulses delivered to electrodes embedded within an implantable device to ionic direct current (iDC) at the output of the device. iDC from the device can then control neural extracellular potential with the intent of being able to not only excite, but also inhibit and sensitize neurons, thereby greatly expanding the possible applications of neuromodulation therapies and neural interface mechanisms. The device of the present invention is designed to reduce power consumption by a factor of 12 and to improve its reliability by a factor of 8.

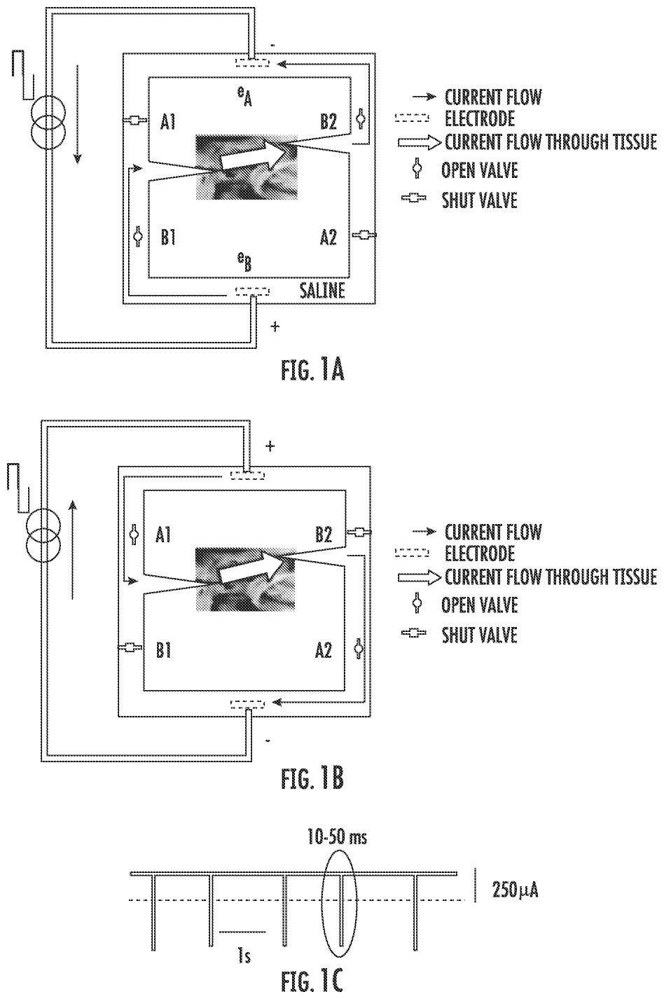

FIGS. 1A and 1B illustrate a schematic diagram of a conceptual SDCS design with two states of the same device. FIG. 1C illustrates a schematic diagram that shows interruptions to output current flow during valve operation. The manufacturability of an implant based on this design is strongly constrained by the high power consumption of mechanical microfluidic valve actuators and the possibilities of the device failure due to the potential failure of any one of its eight mechanical valves. The present invention is directed to an alternative SDCS design, in an effort to improve both power consumption and reliability.

Conceptually, the SDCS delivers alternating current pulses to electrodes suspended at the opposite ends of a torus filled with ionic solution (termed “saline” in FIG. 1A). With each change in stimulation polarity the valves on either side of each electrode change from open-to-closed and closed-to-open, effectively modulating the path for ionic flow through each valve between low impedance and high impedance. Two extension tubes connect to the sides of the torus, such that they can be directed into the body to complete the ionic current circuit. FIGS. 1A and 1B demonstrate this concept comparing the two states of the apparatus. In both the left and the right panels of the figure, the current flows from left to right through the stimulated tissue. In this way, a continuous AC square wave controlling the apparatus will deliver iDC through the tissue from left to right. This system also addresses the problem of ionic buildup, by creating a closed-circuit path for the ions to flow, so that the anions that flow into the electrode tube on the right are replaced by the anions that flow out of the electrode tube on the left.

The fidelity of the DC system output is degraded by periodic interruptions in current flow due to non-ideal behavior of the mechanical valves used in the device (FIG. 1C, indicated by the oval). The interruptions occur because ionic current bypasses the tissue when the valves are temporarily and simultaneously both open or both closed during valve transitions. For example, if A1 and B1 are both temporarily closed during a transition, no current will flow through the tissue. This artifact lasted as long as 50 ms in the original prototype. The degraded fidelity of the direct current flow produced by SDCS1 may be acceptable for acute studies of the SDCS principle of operation (effectively resulting in DC plus a 1 Hz pulsatile stimulus), but smooth flow of DC (or low frequency analog waveform) current without interruptions is required for continuous excitation or inhibition of the target tissue.

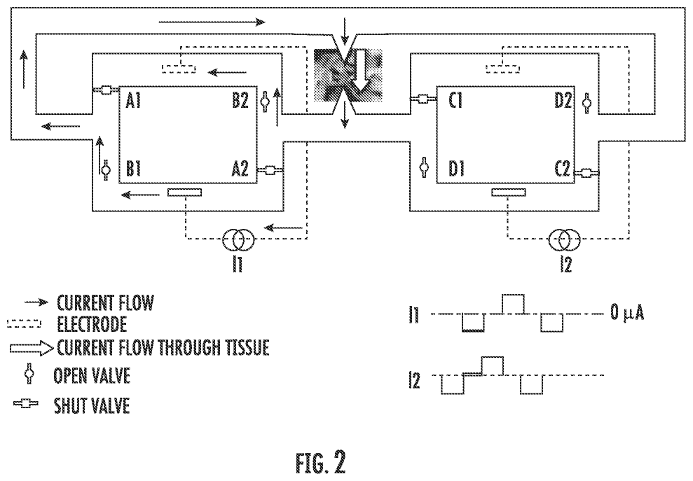

To eliminate DC current flow interruptions another system was developed, which used two SDCS systems in the arrangement shown in FIG. 2. One system drives current through the tissue while the other closes all valves first and then opens the next set of valves in sequence. The intermediate step of closing all valves on the system undergoing valve transitions prevents unintended current shunts through either system. FIG. 2 illustrates a schematic diagram of two system design that uses one system (driven by I1 shown) to drive the current through the tissue, while the second system (driven by I2) to switch the valve states without causing output current interruption.

In the system state shown in FIG. 2, I1 drives the current through the tissue while I2 is shut off. In order to switch valves from open-to-closed and closed-to-open in the right system (I2) from the state depicted in FIG. 2, the D valves are closed first. Because C valves remain shut during this operation, closing D valves will not cause any interruption in current flow even if D valves are relatively slow to close or they do not close at the same instant. Next, C valves are open. This transition does not cause any interruption in current flow, because D valves are now closed. Finally, current control is transitioned to the right system (I2), and simultaneously shut off the left system (I1). Since this transition is electronic rather than mechanical, it is very fast and does not cause interruption in current flow. The procedure is then repeated for the left (I1) system, first closing B valves and then opening A valves, while the right (I2) system drives current through the tissue. In this way, the system shown in FIG. 2 avoids all valve transition artifacts, even when the valves are slow. In attempting to implement this design in a microfluidic substrate challenges centered on the reliability associated with developing eight identical valves, each powered by a separate actuator were encountered. The device of the present invention was developed to resolve these reliability issues.

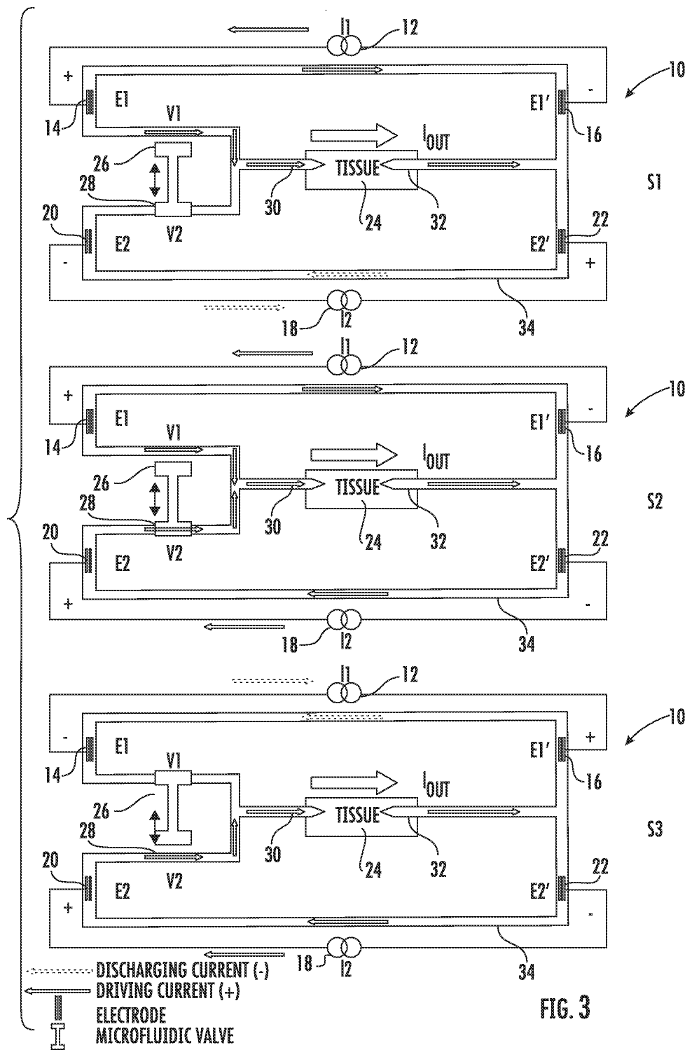

FIG. 3 illustrates a schematic diagram of three states of the present invention. The system 10 cycles from S1 to S2 to S3, and then back from S3 to S2 to S1 continually. During S1, current source I1, 12, drives the current through the tissue via electrodes, E1, 14, and E1′, 16, and current source I2, 18, discharges electrodes E2, 20, E2′, 22. During S3, I2, 18, drives the current through the tissue, 24, and I1, 12, discharges its electrodes. The tandem microfluidic valve is composed of two ports V1, 26, and V2, 28. This V1, 26, and V2, 28, change states during S2 and this transition results in initial opening of both valves and then closing the next one in sequence. The arrows 30, 32, pointing at the tissue represent microcatheter tubes filled with an electrolyte gel to allow ionic current flow to the neural targets. Discharging Current (−) Driving Current (+) Electrode Microfluidic valve S2 S3 S1. Microfluidic channels 34 hold conductive liquid that allows for current to flow from the current sources I1, 12, and I2, 18, to the electrodes.

The construction of the design of the present invention is shown in FIG. 3. The three panels show the varying states of the same device. The blue structures represent the microfluidic channels 34 within the device, filled with an electrolyte. There are four metal electrodes, E1, 14, E1′, 16, E2, 20, and E2′, 22, submerged in the channels. E1, 14, and E1′, 16, are connected via one current source and E2, 20, and E2′, 22, are connected via a second current source. The two current sources I1, 12, and I2, 18 are designed to drive the current through the tissue in sequence, with one current source driving the current indicated in red through the tissue via one set of electrodes, and the other current source driving the current in the opposite direction indicated in black to discharge the electrodes. I1, 12, drives the current from E1, 14, to E1′, 16, passing the current through the tissue in state S1, indicated by grey arrows. The charge builds up on the electrode surfaces and needs to be discharged. This charge is dissipated from E1, 14, and E1′, 16, by changing the state of the device and reversing the current flow through I1, 12, (black arrows) as shown in state S3. In these two states, I2, 18, is driving the current in the opposite direction from I1, 12, with S1 indicating the discharging of E2, 20, and E2′, 22, and S3 showing I2, 18, driving the current through the tissue using E2, 20, and E2, 22.

The microfluidic valve actuator is assumed to be non-ideal and thus take time to switch the state of the device. For this reason, the tandem valve with two ports, V1, 26, and V2, 28, are designed to transition from open-to-closed and closed-to-open in a way that will keep both ports partially open for a short duration during the transition in state S2. During this switch, both current sources I1, 12, and I2, 18 would be driving the current through the tissue. The amplitude of the current during the discharge phase following this transition period is always calculated to account for the total charge accumulated on the electrodes during the driving current phase as the integral of the driving current. To deliver the constant current to the tissue, the device changes states back and forth between S1 and S3 through S2.

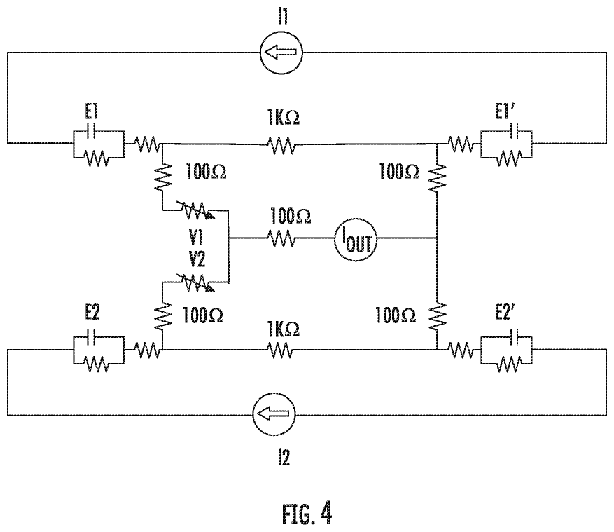

FIG. 4 illustrates a schematic diagram of an electrical equivalent component model of the present invention. The design of the present invention is shown in FIG. 4 using electrical components to represent ionic microfluidic channel impedances, electrode interfaces, and current sources. The tissue is modeled as a 100 kΩ high impedance path due to the narrow-diameter micropipette conduits used to deliver iDC to the neural targets based on previous experiments. The valves are modeled as potentiometers with 1 kΩ conduction path when they are fully open and 10 MΩ when they are completely shut. The electrode contacts are modeled as capacitor/resistor parallel pair with a series resistor. The values for these are 10 μF in parallel with a 2 MΩ resistor that are then in series with a 100Ω. The main assumption for the benefit of the model is that the metal electrodes will have sufficient surface area to avoid Faradaic reactions during device operation. Based on previous experience with microfluidic valve operation, the action of closing and opening the tandem valve in state S2 will take 600 ms. Iout is a measurement of the current delivered to the tissue.

I1 and I2 positive driving currents are designed to overlap during the valve switch transition to maintain the current through the tissue during the transition. The negative discharge currents are designed to rapidly and completely drain the charge accumulated on the electrodes before the next state transition. The current sources are therefore set up to drive 1 mA in the positive direction for four seconds and discharge at 4 mA over one second, thus maintaining charge balance.

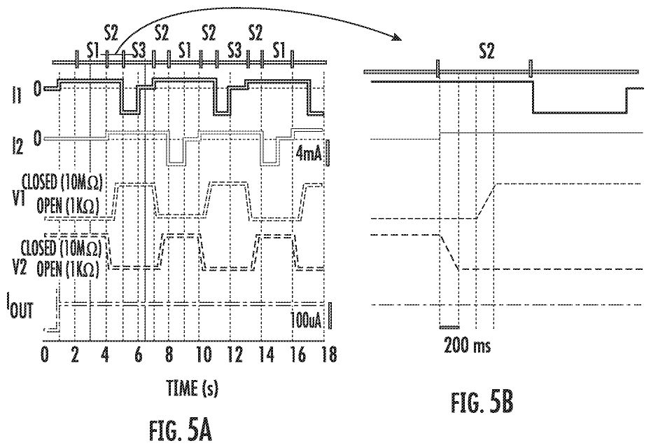

FIGS. 5A and 5B illustrate graphical views of modeling of the present invention. FIG. 5A shows 18 seconds of simulation. FIG. 5B expands the time between 3 and 7 seconds to illustrate the details of the switching sequence time course. The bottom trace of the plot shows the current output to the tissue. Iout appears to be stable in the figure. Upon close examination a 1% undulation in system output is detected when both valves are open during S2.

A new concept is presented herein for the construction of Safe Direct Current Stimulators (SDCS). The goal of any SDCS is to deliver a stable ionic direct current to the neural targets while maintaining charge balance at the metal electrodes embedded within the device. The present invention is directed to an electrical component model, designed to deliver 100 μA to the neural targets. The model results suggest that this design can in principle convert alternating current pulses delivered to metal electrodes to ionic direct current. Current sources I1 and I2 are charge balanced with a stable device output within 1% variance.

The limitations of this model are that it assumes no variability in closed/open valve impedances, in the impedances of the microfluidic channels as well as in the ionic path through the tissue. Any of these variances will clearly affect system output Iout. For this reason, while the basic structure of the design is sound, a control system should be designed to monitor system output, and allow the current sources to compensate for any output irregularities. This control system would adjust the current drivers during the driving phase, integrate the output to determine the exact charge delivered to the electrodes during this time, and during the discharge phase adjust the discharge amplitude to account for the total accumulated charge.

The key features of this new design is that the number of actuators is reduced from eight in the previous embodiment to one in the new design and the number of valves is reduced from eight independent valves to one tandem valve. Because the valve actuator will only need to function once every half-cycle to maintain system state, the amount of energy required to operate the mechanical valve is reduced from the need to operate average of six valves at the same time in SDCS2 to 0.5 duty cycle on one valve, resulting in a factor of 12 improvement in energy consumption. Additional improvement could be made by making the tandem valve bi-stable so the energy applied to the system would only be necessary to transition between S1 and S3, rather than using energy to maintain one system state.

The benefits of this device design is that it uses only one Nitinol wire actuator rather than eight to significantly improve the reliability, and two valves operated in tandem using the single actuator rather than eight valves. The Nitinol wire is energized not for the entire cycle, but only for half the cycle to drive the valves, i.e. V1 is normally open and V2 is normally closed. Therefore instead of six Nitinol wires being engaged to run SDCS2, on average 0.5 wires are being engaged to operate SDCS3. This amounts to 68 mW*0.5=34 mW consumed in the microfluidic valve operation. This operation would therefore consume approximately ⅓ of the ˜100 mA power budget typically consumed by a neural prosthetic system.

The conceptual device construction is shown in FIG. 3, which shows the states of the same device. The device includes the microfluidic channels within the device, filled with an electrolyte. There are four metal electrodes submerged in the channels. Two are connected via one current source and the other two are connected via a second current source. The two current sources I1 and I2 are designed to drive the current through the tissue in sequence, with one current source driving the current indicated in grey through the tissue via one set of electrodes, and the other current source driving the current in the opposite direction indicated in black to discharge the other set of the electrodes. I1 drives the current from E1 to E1′, passing the current through the tissue in the left panel, indicated by grey arrows. However, because the electrode interfaces are large enough to maintain high charge capacity to avoid faradaic reactions, their operation can be thought of as capacitors that build up charge during the current flow. This charge is dissipated from E1 and E1′ by changing the state of the device and reversing the current flow through I1 (black arrows) as shown in the right panel. I2 is always in the opposite state from I1, with the state on the left indicating the discharging of E2 and E2′ and the panel on the right showing I2 driving the current through the tissue using E2 and E2′. A key aspect of the invention is that the mechanism and the sequence of state changes: E1 and E1′ charge up while delivering current to the tissue, while E2 and E2′ are getting discharged, then switch back in a manner that keeps both valves open during the switch to keep the current flow through the tissue from being interrupted.

The Nitinol wire actuator takes up to 0.5 seconds to switch the state of the device. Because the valves are not ideal, both valves will either be open or closed for a short duration during state switch. Simultaneous closure of the valves for any duration is unacceptable because this will cause an automatic interruption in current flow to the tissue. For this reason, the tandem valves are designed to transition from open to close and close to open in a way that will keep both valves partially open for a short duration during the transition. During this switch, both current sources I1 and I2 timing is designed to drive the current through the tissue.

Further details of the design concern the control of the ionic current through the tissue to compensate for variances in valve impedance to ensure that the ionic current delivery is maintained at a steady level. The amplitude of the driving current from the current source that is being turned on next in the sequence is controlled by sensing the amount of ionic current delivered to the tissue (current sensing element (CSE) is described in our previous publications). The amplitude of the current during the discharge phase following this transition period is always calculated as a function of total charge accumulated on the electrodes during the driving current phase, calculated as the integral of the driving current. This ensures that the electrodes never enter the faradaic regime to degrade safety.

Function of the present invention can be carried out in conjunction with a computer, non-transitory computer readable medium, or alternately a computing device or non-transitory computer readable medium incorporated into the medical device associated with the present invention.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can be a special computer designed specifically for this purpose. The computing device can be unique to the present invention and designed specifically to carry out the method and operation of the present invention.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention. While exemplary embodiments are provided herein, these examples are not meant to be considered limiting. The examples are provided merely as a way to illustrate the present invention. Any suitable implementation of the present invention known to or conceivable by one of skill in the art could also be used.

1. A device for safe direct current stimulation of tissue comprising:
an actuator;
a pair of current sources (I1, I2) engaged to apply current by the actuator;
a pair of valves (V1, V2) operated in tandem by the actuator;
a first pair of electrodes (E1 and E2); and,
a second pair of electrodes (E1′ and E2′);
wherein the device is configured to operate in three stages (S1, S2, and S3), such that during S1, I1 drives current through the tissue via E1 and E1′ and I2 discharges E2 and E2′, such that during S2, the valves V1 and V2 change states, such that both valves are first opened and then one of V1 and V2 is closed in sequence, and such that during S3, I2 drives current through the tissue and I1 discharges electrodes E1 and E1′.

2. The device of claim 1, wherein a first microcatheter tube filled with an electrolyte gel is disposed on one side of the tissue and a second microcatheter tube filled with an electrolyte gel is disposed on a second side of the tissue.

3. The device of claim 1, wherein the actuator comprises one NiTiNol wire.

4. The device of claim 3, wherein the NiTiNol wire is energized for half of a cycle to drive the pair of valves.

5. The device of claim 1 further comprising microfluidic channels filled with an electrolyte, wherein the microfluidic channels connect the first pair of electrodes with one of the pair of current sources and wherein the microfluidic channels connect the second pair of electrodes with another one of the pair of current sources.

6. The device of claim 1, wherein contacts of electrodes of the first and second pairs of electrodes take a form of a capacitor/resistor parallel pair with a series resistor.

7. The device of claim 6, wherein the contacts for the electrodes are 10 μF in parallel with a 2 MΩ resistor that are then in series with a 100Ω resistor.

8. The device of claim 1, wherein opening and closing the pair of valves in tandem in state S2 requires 600 ms.

9. The device of claim 1, wherein the pair of current sources are configured to drive current while the pair of valves change state during S2 such that current is maintained through the tissue during the change of state.

10. The device of claim 1, wherein each one of the pair of current sources is configured to drive 1 mA in a positive direction for four seconds and discharge at 4 mA over one second, such that a charge balance is maintained.

11. The device of claim 1, wherein ionic current is directed to the tissue while a charge balance is maintained at the first pair of electrodes and the second pair of electrodes.

12. The device of claim 1, wherein the pair of current sources alternately deliver current pulses to the first and second pair of electrodes, such that alternating current pulses are converted to ionic direct current.

13. A system for safe direct current stimulation of tissue comprising:
an actuator;
a pair of current sources (I1, I2) engaged to apply current by the actuator;
a pair of valves (V1, V2) operated in tandem by the actuator;
a first pair of electrodes (E1 and E2);
a second pair of electrodes (E1′ and E2′);
wherein the device is configured to operate in three stages (S1, S2, and S3), such that during S1, I1 drives current through the tissue via E1 and E1′ and I2 discharges E2 and E2′, such that during S2, the valves V1 and V2 change states, such that both valves are first opened and then one of V1 and V2 is closed in sequence, and such that during S3, I2 drives current through the tissue and I1 discharges electrodes E1 and E1′; and
a control system configured to monitor system output and direct the pair of current sources to compensate for irregularities in output.

14. The system of claim 13, wherein the control system is configured to adjust the pair of current sources during a current driving phase.

15. The system of claim 13, wherein the control system is configured to integrate output of the pair of current sources to determine an exact charge delivered to the first and second pairs of electrodes.

16. The system of claim 13, wherein the control system is configured to discharge amplitude of a charge to account for a total accumulated charge.

17. The system of claim 13, wherein a first microcatheter tube filled with an electrolyte gel is disposed on one side of the tissue and a second microcatheter tube filled with an electrolyte gel is disposed on a second side of the tissue.

18. The system of claim 13, wherein the actuator comprises one NiTiNol wire.

19. The system of claim 13 further comprising microfluidic channels filled with an electrolyte, wherein the microfluidic channels connect the first pair of electrodes with one of the pair of current sources and wherein the microfluidic channels connect the second pair of electrodes with another one of the pair of current sources.

20. The system of claim 13, wherein contacts of electrodes of the first and second pairs of electrodes take a form of a capacitor/resistor parallel pair with a series resistor.
